(19) United States
(12) Patent Application Publication
(10) Pub. No.: US 2021/0045907 A1
(43) Pub. Date: Feb. 18, 2021

(54) Grip buddy

(71) Applicant: Ricardo Ponciano Raquel, San Diego, CA (US)

(72) Inventor: Ricardo Ponciano Raquel, San Diego, CA (US)

(21) Appl. No.: 15/732,256

(22) Filed: Oct. 12, 2017

(51) Int. Cl. A61F 5/01

(52) U.S. Cl. CPC A61F 5/0118

(57) ABSTRACT

A one piece multipurpose device designed to assist people with arthritis and other hand and joint diseases enhance and increase their grip on equipment and tools with little or no discomfort. Secured on the palm of the hand by a single ring that fits loosely around the finger the device is fabricated with liquid silicone rubber. The device can be used on either hand. It maintains its qualities under moist and wet conditions.

A one-piece device primarily invented to help people with arthritis and other related hand and joint disorders to grip tools and equipment with round handles with little or no discomfort.

### CROSS-REFERENCE TO RELATED APPLICATIONS

Not applicable

### BACKGROUND OF THE INVENTION

The device is invented to help people with arthritis and other disabilities of the hands and joints. Enhance their grip on equipment and tools with round handles like golf clubs, garden tools, some medical devices etc.

### BRIEF SUMMARY OF THE INVENTION

The device is a grip aid with many different applications.

### BRIEF DESCRIPTION OF THE DRAWINGS

FIG. 1 is a front view of the device.

FIG. 2 is a side view of the device.

FIG. 3 is a rear view of the device.

FIG. 4 is a top view of the device.

FIG. A is a rear perspective view of the device.

FIG. B is a front perspective view of the device.

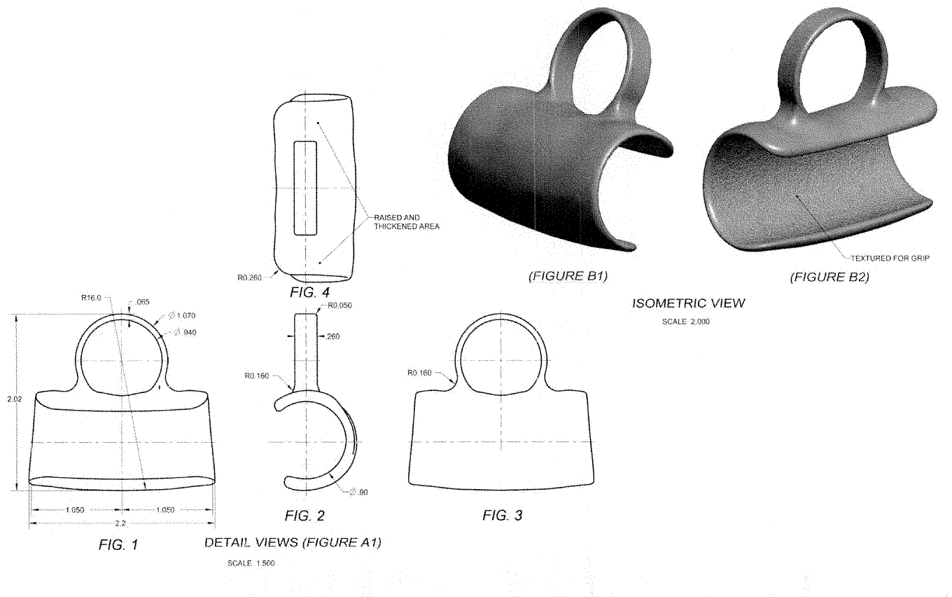

A single round ring on FIGS. 1 and 3 that secures the device on the palm of the hand. a raised and thickened area for cushioning on FIG. 4 designed to absorb impact. Textured to increase gripping capability as shown on figure b2.

### DETAILED DESCRIPTION OF THE INVENTION

A one-piece multi-purpose device fabricated with the use of liquid silicone rubber material which has the properties of having to be biocompatible and hypoallergenic. The device is designed with a single ring that fits loosely around the ring finger or the middle finger. It is loosely designed to be easily position the device on the palm of the hand for comfort. The curled body of the device is positioned on the palm to cradle the equipment or tool as shown on FIG. 2 of drawing. The device can be used on either hand. The device is textured inside to increase the gripping capability as shown on figure B. The device has a raised and thickened area for cushioning to neutralize impact as shown on FIG. 4. The device can be used under moist or wet conditions and still maintain its properties. The device is simple, handy, hygienic, long lasting and effective.

1) A single ring design on a device that enables a user to position the device with ease on the palm of their hand;
wherein the ring fits loosely around a finger (ring or middle finger) so that angles can be manipulated where it is comfortable;
wherein the device comprises a hollow cylinder with a missing section of its side, the cylinder attached to the ring such that the plane of the ring is aligned parallel to a long axis of the cylinder.

2) The device of claim 1, wherein the device is textured on the inside to increase a gripping ability.

3) The device of claim 1, wherein the device is designed with a thickened and raised area to absorb impact and for comfort;
wherein this design to helps with arthritis and other joint problems;
wherein the device is fabricated with liquid silicone rubber that has the properties of having to be biocompatible and hypoallergenic. about 1% or 1 million are allergic to latex rubber.

4)-6). (canceled)
